(19) United States
(12) Patent Application Publication
(10) Pub. No.: US 2023/0320571 A1
(43) Pub. Date: Oct. 12, 2023

(54) ENDOSCOPE INSERTION METHOD AND OVERTUBE

(71) Applicant: OLYMPUS MEDICAL SYSTEMS CORP., Tokyo (JP)

(72) Inventor: Arimasa SUGIMOTO, Hachioji-shi (JP)

(73) Assignee: OLYMPUS MEDICAL SYSTEMS CORP., Tokyo (JP)

(21) Appl. No.: 17/716,119

(22) Filed: Apr. 8, 2022

(51) Int. Cl. A61B 1/00; A61B 1/273; A61B 1/005

(52) U.S. Cl. CPC A61B 1/00135; A61B 1/005; A61B 1/2736; A61B 1/00082

(57) ABSTRACT

An endoscope insertion method and an overtube are disclosed. The method is for inserting an endoscope into the vicinity of a duodenum using an overtube equipped with a raising member, and includes: adjusting an orientation of the distal end of the endoscope protruding from the overtube by the raising member to direct the distal end of the endoscope toward a pylorus; and inserting the distal end of the endoscope into the vicinity of the duodenum. The overtube includes: a tube body; and a first balloon provided on a distal end side of a distal end opening of the tube body. A center of the first balloon in a contracted state and a center of the first balloon in an expanded state are on opposite sides of a central axis of the tube body in an attaching direction of the first balloon to the tube body.

### TECHNICAL FIELD

The present disclosure relates to an endoscope insertion method and an overtube.

### BACKGROUND

A method is used in which an insertion portion of an endoscope is inserted through an overtube and inserted into a luminal organ together with the overtube. In addition, an overtube with a balloon attached to a distal end thereof is used so that the insertion portion of the endoscope can be smoothly inserted into a complicatedly curved gastrointestinal lumen.

When the insertion portion of the endoscope is inserted into the vicinity of the duodenum, the insertion portion of the endoscope protruding from a distal end of the overtube placed in a stomach comes into contact with a stomach wall and easily bends. When the insertion portion of the endoscope comes into contact with the stomach wall and bends, it is difficult to operate the insertion portion of the endoscope.

Specifically, when the insertion portion of the endoscope comes into contact with the stomach wall and bends, it is difficult to transmit an operation of advancing and retreating the insertion portion of the endoscope from an operation portion of the endoscope to the insertion portion. Further, it is difficult to transmit an operation of twisting the insertion portion of the endoscope from the operation portion of the endoscope to the insertion portion. Therefore, for example, problems such as the distal end of the insertion portion inserted into the duodenum unintentionally returning to the stomach are likely to occur.

For example, a known endoscope device includes an overtube including a balloon.

In the known endoscope device, when the insertion portion of the endoscope is inserted into the vicinity of the duodenum, while the balloon provided at the distal end of the overtube is brought into contact with the stomach wall, the distal end of the insertion portion is inserted into the vicinity of the duodenum using the bending function that bends the distal end of the insertion portion of the endoscope. Here, since the distal end of the overtube has a shape along the stomach wall, the insertion portion of the endoscope may come into contact with the stomach wall when the insertion portion of the endoscope is advanced.

### SUMMARY

The present disclosure provides an endoscope insertion method and an overtube that makes it easy to insert an endoscope insertion portion into the vicinity of the duodenum.

The present disclosure proposes the following means.

An endoscope insertion method according to a first aspect of the present disclosure is a method for inserting an endoscope into the vicinity of a duodenum using an overtube equipped with a raising member. The method includes: adjusting an orientation of the distal end of the endoscope protruding from the overtube by the raising member to direct the distal end of the endoscope toward a pylorus; and inserting the distal end of the endoscope into the vicinity of the duodenum.

An overtube according to a second aspect of the present disclosure includes: a tube body through which medical equipment can be inserted; and a first balloon provided on a distal end side of a distal end opening of the tube body. A center of the first balloon in a contracted state and a center of the first balloon in an expanded state are on opposite sides of a central axis of the tube body in an attaching direction of the first balloon to the tube body.

### BRIEF DESCRIPTION OF THE DRAWINGS

FIG. 1 is a diagram showing an endoscope.

FIG. 2 is a diagram showing an overtube according to an exemplary embodiment of the present disclosure.

FIG. 3 is a view of a distal end of the overtube as viewed from a distal end side in a longitudinal direction.

FIG. 4 is a view of the distal end of the overtube as viewed from the distal end side in the longitudinal direction.

FIG. 5 is a diagram showing a first insertion step.

FIG. 6 is a diagram showing a fixing step.

FIG. 7 is a diagram showing an adjustment step.

FIG. 8 is a view of a first balloon expanded in the adjustment step as viewed from the distal end side.

FIG. 9 is a diagram showing a second insertion step.

FIG. 10 is a diagram showing another aspect of the fixing step.

FIG. 11 is a diagram showing an overtube according to an exemplary embodiment of the present disclosure.

FIG. 12 is a cross-sectional view taken along a line X-X of FIG. 11.

FIG. 13 is a diagram showing an adjustment step using the overtube.

### DETAILED DESCRIPTION OF THE PREFERRED EMBODIMENTS

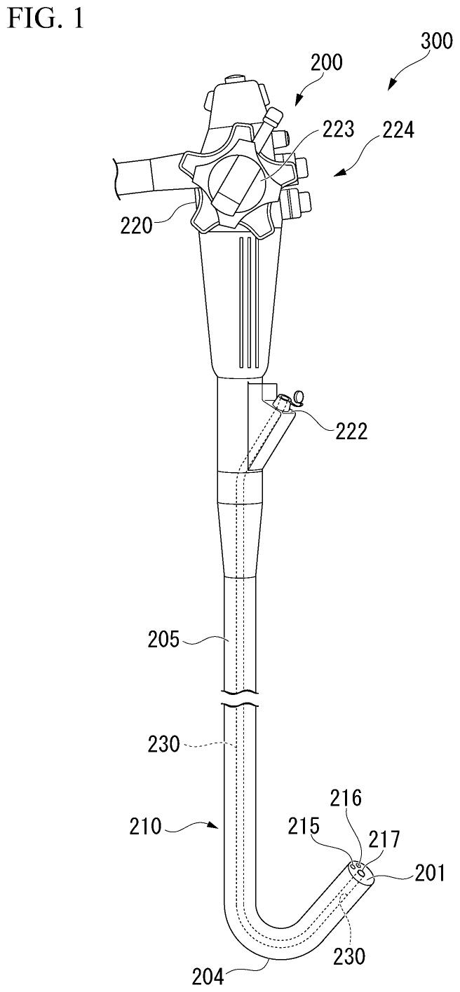

A medical system 300 including an overtube 100 according to an exemplary embodiment of the present disclosure will be described with reference to FIGS. 1 to 9.

[Medical system 300]

The medical system 300 includes an endoscope (medical device) 200 and the overtube 100 through which an insertion portion 210 of the endoscope 200 is inserted. The medical system 300 may be one used by inserting a medical device of a type different from that of the endoscope 200 into the overtube 100.

### [Endoscope 200]

FIG. 1 is a diagram showing the endoscope 200.

The endoscope (medical device) 200 is a known flexible endoscope, and includes the long insertion portion 210 and an operation portion 220 provided on the proximal end side of the insertion portion 210.

The insertion portion 210 has a distal end portion 201, a curved portion 204, and a flexible portion 205. The distal end portion 201, the curved portion 204, and the flexible portion 205 are connected in this order from the distal end of the insertion portion 210. The insertion portion 210 is formed with a treatment tool channel 230 for inserting the treatment tool for endoscopy.

The distal end portion 201 has a light guide 215, an imaging unit 216 having a CCD or the like, and a distal end opening 217 of a treatment tool channel 230.

The curved portion 204 is configured to be freely curved in the vertical direction and the horizontal direction. The distal end of the operation wire is fixed to the distal end side of the curved portion 204. The operation wire extends through the insertion portion 210 to the operation portion 220.

On the proximal end side of the operation portion 220, a knob 223 for operating the operation wire, a switch 224 for operating the imaging unit 216, and the like are provided. The operator can bend the insertion portion 210 in a desired direction by operating the knob 223.

A forceps opening 222 communicating with the treatment tool channel 230 is provided on the distal end side of the operation portion 220. The operator can insert the endoscopic treatment tool through the forceps opening 222.

### [Overtube 100]

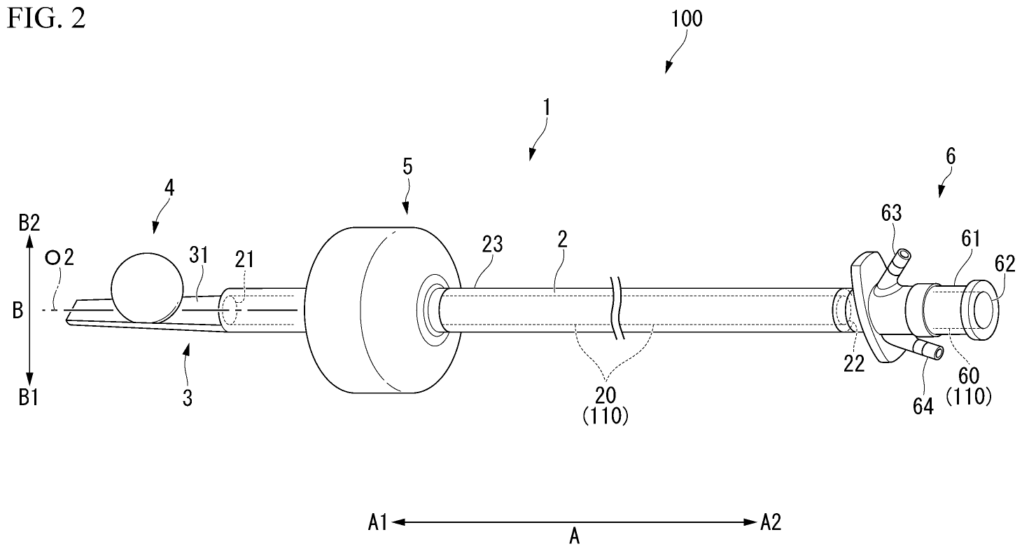

FIG. 2 is a diagram showing an overtube 100.

The overtube 100 is a guide tube having a path 110 through which the insertion portion 210 of the endoscope 200 can be inserted. The operator inserts the insertion portion 210 of the endoscope 200 into the overtube 100, and inserts the insertion portion 210 of the endoscope 200 together with the overtube 100 into a luminal organ such as a stomach or an intestine.

The overtube 100 includes an insertion portion 1 to be inserted into the body and a connection portion 6 attached to the insertion portion 1. The insertion portion 1 includes a main tube (tube main body) 2, a distal end portion 3, a first balloon 4, and a second balloon 5.

In the following description, the side of the distal end portion 3 in the longitudinal direction A of the overtube 100 is the distal end side (distal side) A1 of the overtube 100, and the side of the connection portion 6 is the proximal end side (proximal side) A2 of the overtube 100.

The main tube (tube body) 2 is a flexible tube and has a lumen 20 extending in the longitudinal direction A. The lumen 20 of the main tube 2 is permeable to the insertion portion 210 of the endoscope 200 and forms a part of the path 110. The lumen of the main tube 2 is open in the longitudinal direction A at the distal end opening 21 formed on the distal end side A1. The lumen 20 of the main tube 2 is opened in the longitudinal direction A at the proximal end opening 22 formed on the proximal end side A2.

The distal end portion 3 is a plate-shaped member extending in the longitudinal direction A, and is attached to the edge of the distal end opening 21 of the main tube 2. The distal end portion 3 may have a flat plate shape or a curved plate shape. The distal end portion 3 may be integrally formed with the main tube 2.

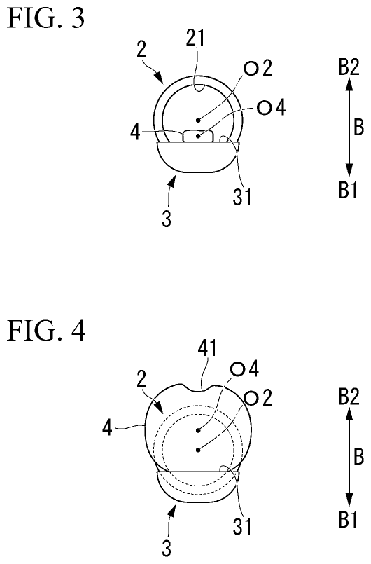

FIGS. 3 and 4 are views of the distal end portion 3 as viewed from the distal end side A1 in the longitudinal direction A.

The distal end portion 3 is provided at a position that does not overlap with the central axis O2 of the main tube 2 when viewed from the longitudinal direction A. The distal end portion 3 is provided on only one side of both sides of the central axis O2. In the following description, one side of both sides of the central axis O2 where the distal end portion 3 is provided is referred to as a lower side (first direction) B1 and the other side is referred to as an upper side (second direction) B2.

The first balloon 4 is an expandable and contractible balloon provided on the distal end side A1 of the distal end opening 21. The first balloon 4 is attached to the upper surface 31 which is the surface of the upper side B2 of the distal end portion 3. The first balloon 4 shown in FIGS. 1 and 3 is in a contracted state. The center O4 of the first balloon 4 in the contracted state is arranged on the lower side B1 of the central axis O2. As shown in FIG. 3, it is desirable that the entire first balloon 4 in a contracted state is arranged on the lower side B1 of the central axis O2. The first balloon 4 may be housed in a contracted state due to elastic deformation, or may be housed in a folded state.

The first balloon 4 shown in FIGS. 2 and 4 is in an expanded state. The first balloon 4 in the expanded state is substantially spherical, and at least a part thereof extends to the upper side B2 of the central axis O2. As shown in FIG. 4, it is desirable that the center O4 of the first balloon 4 in the expanded state is located on the upper side B2 of the central axis O2. The first balloon 4 has a recess 41 arranged in the upper side B2 in the expanded state.

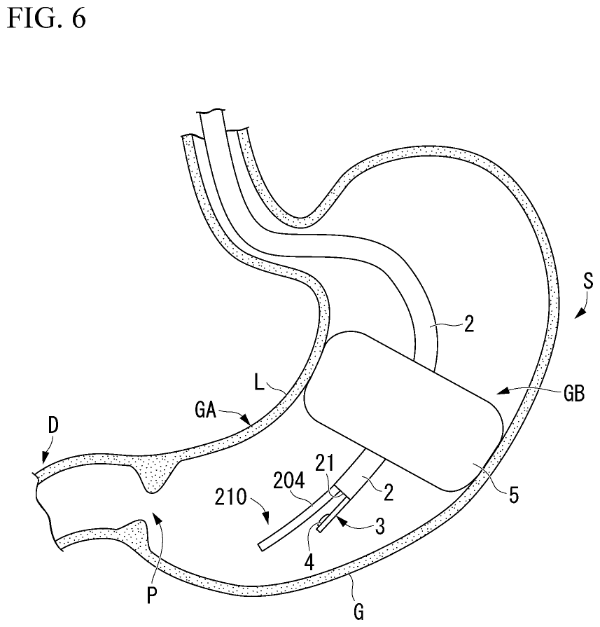

The second balloon 5 is an expandable and contractible balloon provided on the proximal end side A2 of the distal end opening 21. The second balloon 5 is attached to the outer peripheral portion 23 of the main tube 2 on the distal end side A1 of the main tube 2. The second balloon 5 shown in FIG. 1 is in a contracted state. The second balloon 5 shown in FIG. 2 is in an expanded state. The second balloon 5 in the expanded state is a substantially ellipsoid. The central axis of the second balloon 5 in the expanded state is coaxial with the central axis O2 of the main tube 2. The second balloon 5 is expandable to the extent that it touches both sides of the body body GB or the angle GA of the stomach S (see FIG. 6). The central axis of the second balloon 5 in the expanded state does not have to be coaxial with the central axis O2 of the main tube 2, and may be eccentric with respect to the central axis O2 of the main tube 2.

The connection portion 6 is attached to the proximal end side A2 of the main tube 2, and has a tubular portion 61, a first air supply connection portion 63, and a second air supply connection portion 64.

The tubular portion 61 has a substantially cylindrical shape coaxial with the main tube 2. The lumen 60 of the tubular portion 61 communicates with the lumen 20 of the main tube 2 via the proximal end opening 22 at the distal end side A1. The lumen 60 of the tubular portion 61 is open in the longitudinal direction A at the insertion port 62 formed on the proximal end side A2.

The first air supply connection portion 63 is a connector that is detachably connected to the air supply source. The first air supply connection portion 63 is connected to the first balloon 4 with a first air supply line (not shown). The first air supply line is a line for sending and removing air to and from the first balloon 4.

The second air supply connection portion 64 is a connector that is detachably connected to the air supply source. The second air supply connection portion 64 is connected to the second balloon 5 with a second air supply line (not shown). The second air supply line is a line for sending and removing air to and from the second balloon 5.

### [Treatment Using Medical System 300]

Next, the treatment using the medical system 300 will be described. Specifically, a method of inserting the endoscope 200 into the vicinity of the pylorus P of the stomach S or the duodenum D and a method of treating the vicinity of the pylorus P of the stomach S or the duodenum D by endoscopic treatment will be described.

### <First Insertion Step S1>

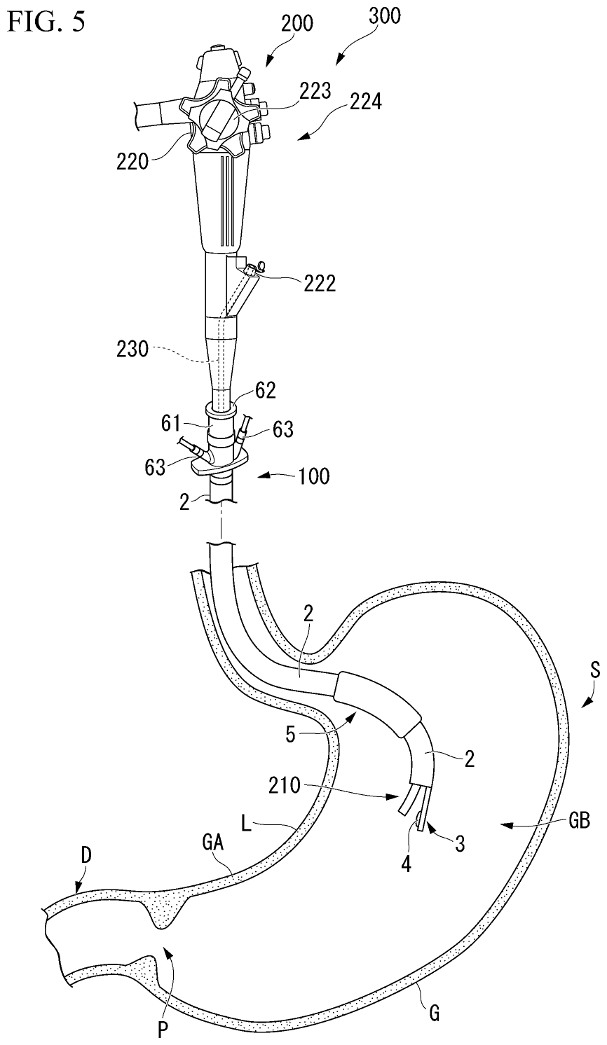

FIG. 5 is a diagram showing a first insertion step.

The operator inserts the distal end of the endoscope 200 and the distal end of the overtube 100 into the stomach S (first insertion step). The operator inserts the insertion portion 210 of the endoscope 200 into the stomach S in a state of being inserted through the path 110 of the overtube 100. The operator may insert the distal end of the endoscope 200 into the stomach S by inserting the distal end of the overtube 100 into the stomach S and then inserting the insertion portion of the endoscope 200 into the path 110 of the overtube 100.

<Fixing step S2>

FIG. 6 is a diagram showing a fixing step.

After the first insertion step, the operator fixes the overtube 100 to the stomach S by inflating the second balloon 5 (fixing step). Specifically, the operator brings the inflated second balloon 5 into contact with the gastric corpus GB or the gastric corpus GA of the stomach S and fixes it. The operator fixes the overtube 100 to the stomach S so that the distal end portion 3 of the overtube 100 is placed on the side of the greater curvature G instead of the side of the lesser curvature L.

By the fixing step, the overtube 100 is fixed in contact with the gastric corpus GB or the gastric corpus GA of the stomach S. As a result, as shown in FIG. 6, the distal end opening 21 of the main tube 2 faces the curvature G rather than the pylorus P.

### <Adjustment Step S3>

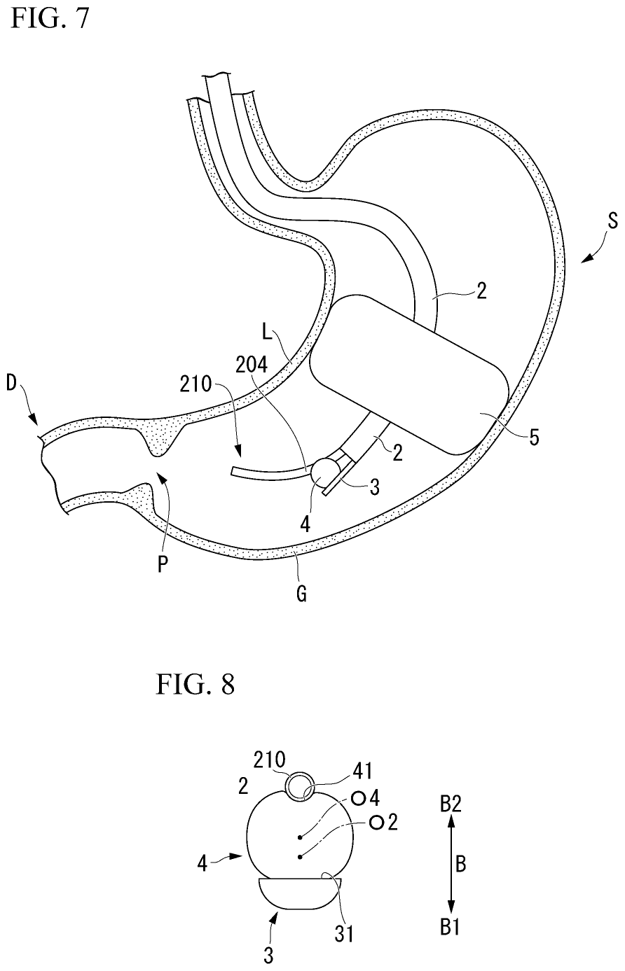

FIG. 7 is a diagram showing an adjustment step.

After the fixing step, the operator directs the distal end of the endoscope 200 protruding from the distal end opening 21 of the overtube 100 toward the pylorus P by the first balloon 4 (adjustment step). Specifically, the operator protrudes the distal end of the endoscope 200 from the distal end opening 21 of the overtube 100. Next, the operator operates the knob 223 of the operation portion 220 so that the curved portion 204 is not subjected to the force for bending the curved portion 204 from the operation portion 220 (neutral state). The curved portion 204 in the neutral state has a straight shape if it is not affected by an external force. Next, as shown in FIG. 7, the operator inflates the first balloon 4 while contacting the insertion portion 210 (for example, the curved portion 204) of the endoscope 200 with respect to the first balloon 4.

FIG. 8 is a view of the first balloon 4 expanded in the adjustment step as viewed from the distal end side A1.

The center O4 of the first balloon 4 in the expanded state moves to the upper side B2 of the central axis O2. The insertion portion 210 of the endoscope 200 arranged near the central axis O2 moves to the upper side B2 by coming into contact with the expanding first balloon 4. In the first balloon 4, since the recess 41 provided in the upper side B2 engages with the insertion portion 210 of the endoscope 200, the insertion portion 210 of the endoscope 200 can be suitably moved with respect to the upper side B2.

By the adjustment step, the insertion portion 210 (for example, the curved portion 204) of the endoscope 200 is curved, and the distal end of the endoscope 200 is directed toward the pylorus P. The operator may adjust the size of the first balloon 4 so that the distal end of the endoscope 200 faces the pylorus P more.

### <Second Insertion Step S4>

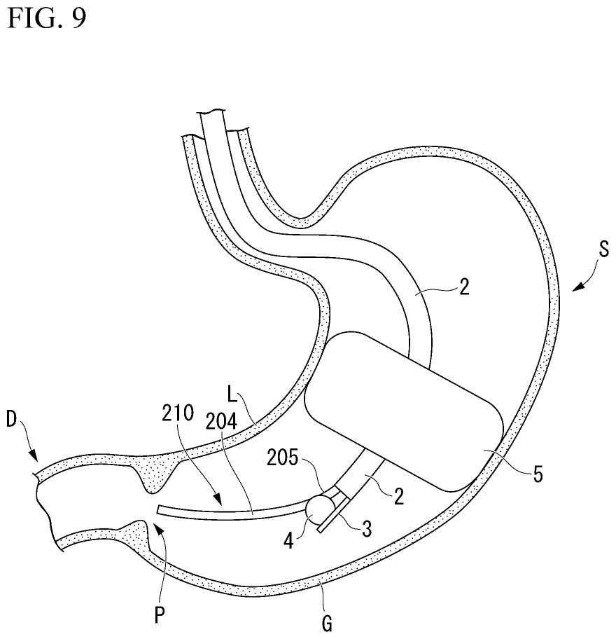

FIG. 9 is a diagram showing a second insertion step.

After the adjustment step, the operator advances the endoscope 200 with respect to the overtube 100, passes the distal end of the endoscope 200 through the pylorus P, and inserts it into the duodenum D (second insertion step). The first balloon 4 comes into contact with the soft portion 205 of the endoscope 200. Since the curved portion 204 is in a state of not receiving a force for bending the curved portion 204 from the operation portion 220 (neutral state), the curved portion 204 that moves forward and is separated from the first balloon 4 has a straight shape. Therefore, when the insertion portion 210 is inserted into the duodenum D, it is difficult to come into contact with the stomach wall of the stomach S.

Even if the distal end of the endoscope 200 does not accurately face the pylorus P, if it roughly faces the pylorus P, the operator can easily insert the distal end of the endoscope 200 into the pylorus P by bending the curved portion 204 by the operation portion 220 and moving the endoscope 200 forward and backward. When treating the vicinity of the pylorus P, the operator may bring the distal end of the endoscope 200 closer to the pylorus P without passing through the pylorus P.

### <Treatment Step S5>

After the second insertion step, the operator treats the affected part of the pylorus P of the stomach S or the affected part of the duodenum D by endoscopic treatment. Since the insertion portion 210 of the endoscope 200 is not in contact with the stomach wall of the stomach S, the operator can smoothly transmit the operation of moving the insertion portion 210 forward and backward and the operation of twisting the insertion portion 210 from the operation portion 220 to the insertion portion 210, and preferably perform endoscopic treatment.

According to the method of inserting the endoscope 200 using the overtube 100 according to the present embodiment, the insertion portion 210 can be inserted near the pylorus P of the stomach S or into the duodenum D in a state where the curved portion 204 is not subjected to a force for bending the curved portion 204 from the operation portion 220 (neutral state). Therefore, it is possible to suitably prevent the insertion portion 210 from coming into contact with the stomach wall of the stomach S and bending the insertion portion 210. Further, the operation of moving the insertion portion 210 forward and backward and the operation of twisting the insertion portion 210 are suitably transmitted from the operation portion 220 to the insertion portion 210.

Although the above embodiment has been described in detail with reference to the drawings, the specific configuration is not limited to this embodiment and includes design changes and the like within a range not deviating from the gist of the present disclosure. In addition, the components shown in the above-described embodiment and the modifications shown below can be appropriately combined and configured.

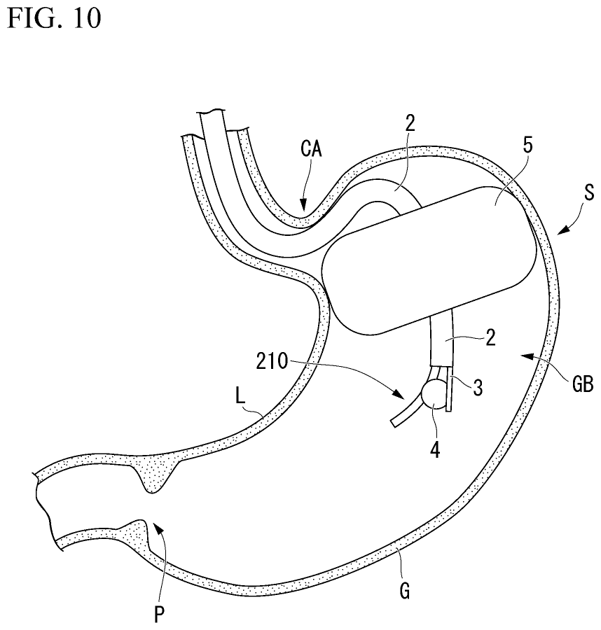

FIG. 10 is a diagram showing another aspect of the fixing step.

In the fixing step of the above embodiment, when the insertion portion 210 is advanced but not inserted into the duodenum D, the second balloon 5 may be re-fixed at the side of the cardia CA so that the distal end portion 3 of the overtube 100 is arranged in the central portion of the stomach S as shown in FIG. 10.

The operator may perform the adjustment step again after the second insertion step. The adjustment step to be performed again is also referred to as a “readjustment step”. Even if the insertion portion 210 of the endoscope 200 is advanced in the second insertion step, the distal end of the endoscope 200 may not face the pylorus P. In particular, as shown in FIG. 10, when the distance between the distal end portion 3 of the overtube 100 and the pylorus P is large, this phenomenon is likely to occur. In this case, the operator inflates (or contracts) the first balloon 4 again and adjusts the distal end of the endoscope 200 so that the distal end of the endoscope 200 faces the pylorus P (readjustment step).

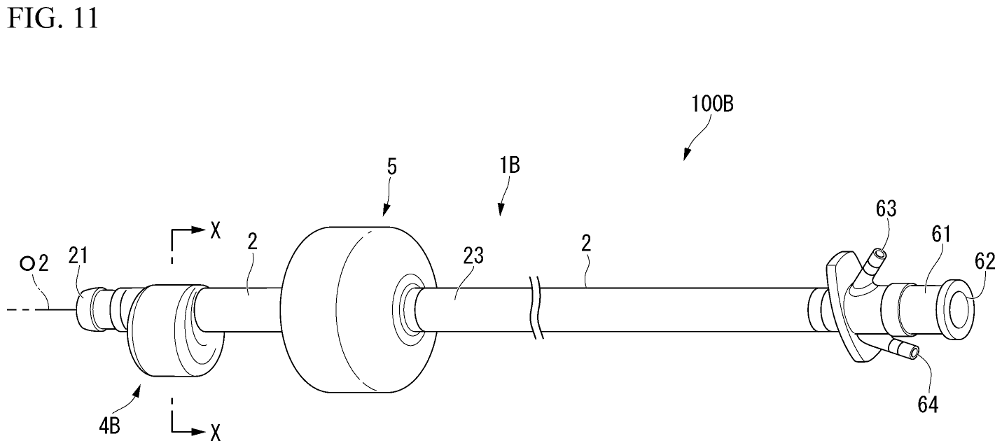

The overtube 100B according to another exemplary embodiment of the present disclosure will be described with reference to FIGS. 11 to 13. In the following description, the same reference numerals will be provided to the configurations common to those already described, and duplicate descriptions will be omitted. The overtube 100B according to the present embodiment is different from the overtube 100 according to the above embodiment in the first balloon 4.

### [Overtube 100B]

FIG. 11 is a diagram showing an overtube 100B.

The overtube 100B is a guide tube having a path 110 through which the insertion portion 210 of the endoscope 200 can be inserted, similarly to the overtube 100 of the above embodiment.

The overtube 100B includes an insertion portion 1B to be inserted into the body and a connection portion 6 attached to the insertion portion 1B. The insertion portion 1B includes a main tube (tube body) 2, a first balloon 4B, and a second balloon 5.

Like the second balloon 5, the first balloon 4B is an expandable and contractible balloon provided on the proximal end side A2 of the distal end opening 21. The first balloon 4B is attached to the outer peripheral portion 23 of the main tube 2 on the distal end side A1 with respect to the second balloon 5.

FIG. 12 is a cross-sectional view taken along the line X-X of FIG. 11.

The first balloon 4B is attached to a part 25 in the circumferential direction of the outer peripheral portion 23 of the main tube 2. Therefore, the center O4 of the first balloon 4B in the expanded state shown in FIG. 12 is eccentric with respect to the central axis O2 of the main tube 2.

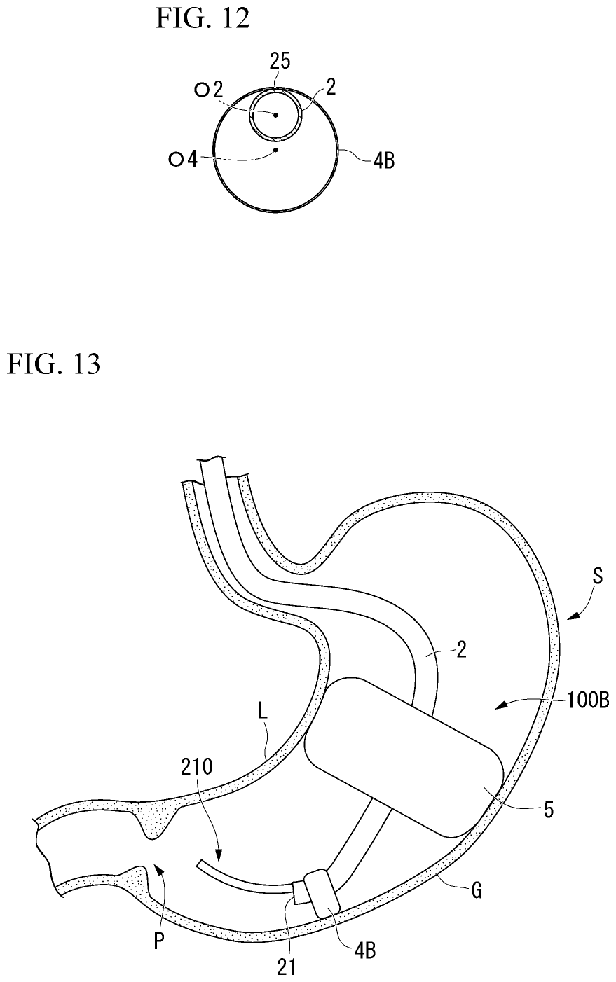

FIG. 13 is a diagram showing an adjustment step using the overtube 100B.

The operator expands the first balloon 4B and brings it into contact with the large swelling G of the stomach S, and directs the distal end opening 21 of the main tube 2 toward the pylorus P. Thereby, the operator can insert the insertion portion 210 into the vicinity of the pylorus P of the stomach S or the duodenum D in a state where the curved portion 204 is not subjected to the force for bending the curved portion 204 from the operation portion 220 (neutral state).

According to the method of inserting the endoscope 200 using the overtube 100B according to the present embodiment, the insertion portion 210 can be inserted near the pylorus P of the stomach S or into the duodenum D in a state where the curved portion 204 is not subjected to a force for bending the curved portion 204 from the operation portion 220 (neutral state).

Although the present embodiment has been described in detail with reference to the drawings, the specific configuration is not limited to this embodiment and includes design changes and the like within a range not deviating from the gist of the present disclosure. In addition, the components shown in the above-described embodiment and the modifications shown below can be appropriately combined and configured.

In the above embodiment, the orientation of the insertion portion 210 of the endoscope 200 is adjusted by the first balloons (raising members) 4, 4B. However, the the raising member that changes the direction of the insertion portion 210 is not limited to this. The raising member may be a lever, a pulley, or the like.

What is claimed is:

1. A method for inserting an endoscope into a vicinity of a duodenum using an overtube equipped with a raising member, the method comprising:
inserting a distal end of the endoscope and a distal end of the overtube into a stomach;
fixing the overtube to the stomach;
adjusting an orientation of the distal end of the endoscope protruding from the overtube by the raising member to direct the distal end of the endoscope toward a pylorus; and
inserting the distal end of the endoscope into the vicinity of the duodenum.

2. The method according to claim 1, wherein
the raising member is a first balloon, and
the adjusting of the orientation of the distal end of the endoscope includes directing the distal end of the endoscope toward the pylorus while inflating the first balloon.

3. The method according to claim 1, wherein
the raising member is a first balloon, and
the adjusting of the orientation of the distal end of the endoscope includes inflating the first balloon while bringing the endoscope into contact with the first balloon.

4. The method according to claim 1, wherein
the endoscope includes an operation portion and a curved portion that is configured to be bent by a force from the operation portion, and
during the adjusting of the orientation of the distal end of the endoscope, the curved portion of the endoscope is not subjected to the force for bending the curved portion from the operation portion of the endoscope.

5. The method according to claim 1, wherein
the fixing of the overtube to the stomach includes fixing the overtube to the corpus or angular incisure of the stomach.

6. The method according to claim 1, wherein
the overtube further includes a second balloon, and
the fixing of the overtube to the stomach includes fixing the overtube to the stomach by inflating the second balloon.

7. The method according to claim 6, further comprising:
repositioning the second balloon and re-fixing the overtube to the stomach after the adjusting of the orientation of the distal end of the endoscope.

8. The method according to claim 2, further comprising:
expanding or contracting the first balloon to reorient the distal end of the endoscope protruding from the overtube toward the pylorus after the distal end of the endoscope is inserted into the vicinity of the duodenum.

9. An overtube comprising:
a tube body through which medical equipment can be inserted, the tube body extending along a central axis from a proximal end to a distal end; and
a first balloon provided on a distal end side of a distal end opening of the tube body, an attaching direction of the first balloon to the tube body being orthogonal to the central axis,
wherein a center of the first balloon is provided on:
a first side of the central axis of the tube body in the attaching direction when the first balloon is in a contracted state, and
a second side of the central axis of the tube body in the attaching direction when the first balloon is in an expanded state, the second side being on an opposite side of the central axis than the first side in the attaching direction.

10. The overtube according to claim 9, further comprising:
a second balloon on a proximal end side of the first balloon and on an outer peripheral portion of the tube body.

11. The overtube according to claim 9, wherein an entirety of the first balloon in the contracted state is arranged on the first side of the central axis of the tube body in the attaching direction.

12. The overtube according to claim 9, wherein the first balloon in the expanded state includes a recess arranged on the second side in the attaching direction.
